(19) United States
(12) Patent Application Publication
(10) Pub. No.: US 2024/0157458 A1
(43) Pub. Date: May 16, 2024

(54) WELDING HELMET

(71) Applicant: BAE SYSTEMS plc, London (GB)

(72) Inventors: Norman Wharton, Cumbria, Barrow-in-Furness (GB); Lee Wagstaff, Cumbria, Barrow-in-Furness (GB); Lee Shaun Waplington, Cumbria, Barrow-in-Furness (GB); Martin Flynn, Cumbria, Barrow-in-Furness (GB)

(73) Assignee: BAE SYSTEMS plc, London (GB)

(21) Appl. No.: 18/552,001

(22) PCT Filed: Mar. 11, 2022
(86) PCT No.: PCT/GB2022/050632

§ 371 Date: Sep. 22, 2023

(30) Foreign Application Priority Data
Mar. 22, 2021 (GB) 2103911.0

(51) Int. Cl. B23K 9/095; A61F 9/06; B23K 9/32

(52) U.S. Cl. CPC B23K 9/095; A61F 9/06; B23K 9/32

(57) ABSTRACT

The present invention relates to improvements in the active welding helmets, specifically for providing welding information to the user. There is provided an active welding helmet for a user welding components, comprising a welding helmet, wherein the helmet comprises at least one operational parameter monitor, operably linked to an indicator, wherein said indicator provides an output stimulus to the user related to an operational parameter.

The present invention relates to improvements in welding helmets, specifically for providing an active welding helmet to provide information to the user.

Welding helmets provide protection to the user during the process of welding. They comprise a face guard to protect the face, neck and eyes. To allow the user to view the weld, and avoid damage to the eye, a specific lens shade is used within the guard, through which the user views the welding process. The lens shade has a shade/tint which can be selected or may be light sensitive in order to protect the user's eyes against intense light, UV and IR emitted by the welding torch.

According to a first aspect of the present invention, there is provided an active welding helmet for a user welding components, comprising a welding helmet, wherein the helmet comprises at least one operational parameter monitor, operably linked to an indicator, wherein said indicator provides an output stimulus to the user related to an operational parameter; as provided by the at least one operational parameter monitor.

The use of operational parameter monitors integrally formed as part of the welding helmet allows the user to only receive information from the helmet. In typical welding process the use of separate devices are often used, which may be located remote from the user, and involve looking away from the welding process.

Preferably, one of the at least one operational parameter monitors may be a thermocouple or non-contact thermometer to measure the operational parameter of the temperature of a component to be welded, wherein the indicator provides the user with the temperature of the component to be welded. Preferably, a non-contact thermometer.

It is essential in many welding process to ensure that the components to be welded are pre-heated to a specific temperature to avoid stresses being introduced to the final welded component. Typically the temperatures are measured using colour changing contact strips etc.

The non-contact thermometer may preferably be an IR laser thermometer. There may be at least two, or at least three non-directional laser thermometers, which are located on the front of the helmet arranged to provide a fixed focus point, at defined distance from the helmet. The focus point being the component to be welded. The use of non-directional lasers may require the average temperature to be used to ensure that they are all focused on the component to be welded. The laser thermometers typically have a visible aiming dot or pattern to show the user where the thermometer is taking its measurement, the user can move their head to point the helmet in the correct direction.

The IR laser thermometer may be a scanning laser thermometer, such that the temperature of dedicated zone or region in front of the user is determined, this may relieve the burden on the user to aim the non-contact thermometer.

In a highly preferred arrangement there is provided an active welding helmet for a user welding components, comprising a welding helmet, wherein the helmet comprises a temperature monitoring laser to monitor the temperature of a component to be welded, an indicator to provide an output related to operational parameters, said indicator operably linked to the temperature monitoring laser, to provide the user with the temperature of the component to be welded.

The operational parameter may be any parameter associated with the safety of performing the process, improving the efficiency of the process and/or improving the final quality of the final welded component. An example of monitoring safety may be where one of the at least one monitors is an atmospheric analyser, located inside the helmet, such as to measure the operational parameter of the user's ambient temperature. The atmospheric analyser may also detect the presence of noxious gases emitted during the welding process.

The welding process typically generates large amounts of heat, both from the pre-welding heating of the component and also from the use of the welding torch. The user can readily experience heat fatigue, especially in confined spaces, hence the health of the user may be protected by ensuring they do not overheat.

Further biometric operational parameters may also be recorded, such as, for example blood pressures, oxygen sats., heart rates, and may be used to assess the health of the user during the welding process.

The monitors may be connected via wires to an industrial user interface system, or directly to the indicators, or may be connected wirelessly.

The indicator may provide a stimulus that is outputted to the user, to alert them to the presence of an operational parameter, or to the change of an operational parameter, such as, for example an audio stimulus, visual stimulus or vibratory stimulus or combinations thereof to the user. In a preferred arrangement the indicator is a head-up display, which can provide a visual output of the at least one or plurality of operational parameter.

Conveniently, there may be a plurality of channels, each associated with each of said operational parameters. In one arrangement, each channel may have a different selected audio stimulus, visual stimulus or vibratory stimulus.

There may be wireless communication capability for each of said channels.

Where the indicator's output stimulus is other than a HUD, the output stimulus may undergo a user determinable change to correlate with a change in said operational parameter(s). For example the frequency and/or amplitude of an audible sound may change with the nearing towards a threshold value for the operational parameter.

The operational parameters may have minimum and/or maximum threshold levels, such that if at least one operational parameter is outside of said threshold it may cause activation of the indictor, or cause a determinable change to the output stimulus of the indicator. If the threshold parameter is safety critical, to the equipment or health of the user, the welding apparatus may be deactivated. This may include stopping the welding process such as by turning off power or fuel supply to the welding apparatus.

The welding helmet as defined herein may have an integral battery power source. The helmet may further comprise a data logger to record the operational parameters. This history may be useful to determine process efficiency. The data logger may be interrogated at the end of a process, or may be continuously monitored remotely via a wireless solution to a dedicated monitoring system. Preferably the welding helmet data is managed by an industrial user interface system. The input channels from the sensors and output channels which drive the indicators are managed by the industrial user interface system to manage said operational parameters, such as, for example PanelPilotACE design studio. The multi-channel panel meter is a human machine interface which allows the control of multiple channel inputs both wireless and wired. The industrial user interface may comprise a touch screen, and associated development board(s) capable of receiving a plurality of inputs and providing channels for outputs. The industrial user interface may be software driven to allow the user to determine threshold limits of the operational parameters, and to allow a welding process to be logged and analysed.

The industrial user interface system may be worn by the user, or may be remote from the user within wireless communication range of the welding helmet. The wireless system may be any communication standard such as Wi-Fi, Bluetooth, and NFC etc. There may be provided a welding helmet system, comprising the welding helmet as defined herein and further comprising the industrial user interface system to manage said operational parameters. The industrial user interface system may receive temperature measurements from a non-contact thermometer and/or a thermocouple, the latter being directed affixed to the component(s) to be welded.

For a better understanding of the invention, and to show how arrangements of the same may be carried into effect, reference will now be made, by way of example, to the accompanying diagrammatic drawings in which:

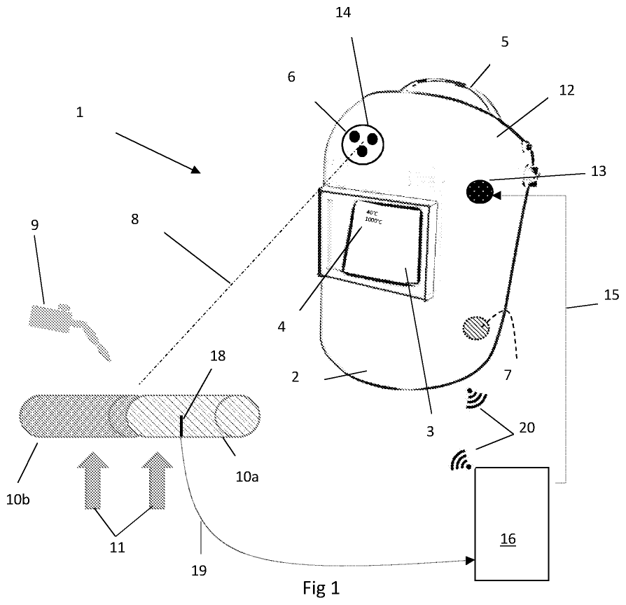

FIG. 1 shows welding helmet according to the invention.

Turing to FIG. 1 there is provided a welding system 1, with a welding helmet 12 which comprises a shading lens 3 that provides the user with protection against harmful UV and high intensity light, whilst retaining the ability to view the welding process. The helmet may be a simple face guard 2 with a head strap 5, or it may be a full re-entrant helmet (akin to a motorbike helmet), which covers the user's entire head. The helmet 12 comprises a plurality of operational parameter monitors 14, which detect the environment proximate to the user or the components to be welded etc. The use of a non-contact laser thermometer 6 may provide the temperature of the components to be welded 10a, 10b, i.e. the pre-weld temperature. The components 10a, 10b, are heated 11 to a tightly defined temperature, such as by inductive heating or a flame. Once the pre-weld temperature has been reached the non-conductive thermometer 6 will detect 8 the temperature and indicate the temperature via indicator 4 (a head up display), that the desired temperature has been reached. The indicator 4, may alternatively be an audible sound or a vibration stimulus that alerts that the pre-weld temperature has been reached.

The user (not shown) may experience significant thermal fatigue, from operating the welding torch 9 and/or being proximate to the heated components 10a, 10b. The helmet may have an atmospheric analyser 7, located inside the helmet, such as to measure the operational parameter of the user's ambient temperature and/or presence of noxious gases. The user's temperature and any gases may be displayed on the HUD, any dangerous threshold levels for gases, or temperatures may be also cause a further emergency indicator i.e. a sound or vibration from speaker 13 and may be programmed to automatically disengage the welding torch 9 and/or any heat source 11 for the pre-welding heating. The helmet system may comprise an industrial user interface 16 to manage said operational parameters from the sensors 14, 6, 7. The industrial user interface system may receive the temperature measurements from a non-contact thermometer 6, wirelessly 20, and/or may receive 19 temperature measurements via a thermocouple 18, affixed to the component(s) to be welded 10a, 10b. The industrial user interface 16, may provide the required output signals to the indicators, such as the speaker 13 and the HUD 4. The industrial user interface 16 may be operably linked to the welding torch 9, such that after a series of warning indicators 14 have been communicated to the user, with changes in urgency such as change of amplitude, frequency or pitch or a flashing message on the HUD 4, that once a threshold has been breached the industrial user interface 16 may automatically cause the shut off of power and or fuel to the welding torch 9 or prevent the heating 11 to the components to be welded 10a, 10b. Other users nearby may also be alerted, or a centralised control facility may be alerted to the breach of threshold values.

1. An active welding helmet for a user welding components, comprising a welding helmet, wherein the helmet comprises an operational parameter monitor, operably linked to an indicator, wherein said indicator provides an output stimulus to the user, the output stimulus related to an operational parameter.

2. The active welding helmet according to claim 1, wherein the operational parameter monitor is a non-contact thermometer or thermocouple, and the operational parameter is temperature of a component to be welded, and the non-contact thermometer or thermocouple is configured to measure the temperature of a component to be welded, and the indicator provides the user with the temperature of the component to be welded.

3. The active helmet according to claim 2, wherein the non-contact thermometer is an IR laser thermometer.

4. The helmet according to claim 1, wherein the operational parameter monitor is an atmospheric analyser, located inside the helmet.

5. The helmet according to claim 1, wherein the output stimulus provided by the indicator includes an audio stimulus, a visual stimulus, or a vibratory stimulus, or combinations thereof, to the user.

6. The helmet of claim 1, wherein the indicator is a head-up display.

7. The helmet of claim 1, wherein the operational parameter monitor is one of a plurality of operational parameter monitors, the helmet comprising a plurality of channels, each channel associated with one or more of said operational parameter monitors.

8. The helmet according to claim 1, wherein the output stimulus undergoes a user determinable change to correlate with a change in said operational parameter.

9. The helmet according claim 7, wherein each channel has a different selected audio stimulus, visual stimulus, or vibratory stimulus.

10. The helmet of claim 1, comprising an integral battery power source.

11. The helmet according to claim 1, comprising an industrial user interface system.

12. The helmet according to claim 11, wherein the industrial user interface system receives inputs from the operational parameter monitor and determines the output to the indicator, to manage said operational parameter.

13. The helmet according to claim 7, comprising wireless communication capability for each of said channels.

14. The helmet according claim 1, wherein the operational parameter is associated with a threshold level, such that if the operational parameter is outside of said threshold, a welding torch is automatically deactivated.

15. The helmet according to claim 4, wherein the operational parameter includes an ambient temperature of the user, and the operational parameter monitor is configured to measure the user's ambient temperature.

16. The helmet according to claim 4, wherein the operational parameter includes a level of a noxious gas inside the helmet, and the operational parameter monitor is configured to detect the level of the noxious gas inside the helmet.

17. An active welding helmet for a user welding components, the helmet comprising:
a welding helmet including a shading lens;
a first sensor, within the welding helmet, and configured to detect an ambient temperature of the user;
a second sensor, within the welding helmet, and configured to detect a level of noxious gas present within the helmet;
a first indicator configured to provide a first output stimulus to the user, the first output stimulus related to one or both of the ambient temperature of the user and the level of noxious gas present within the helmet; and
a second indicator configured to provide a second output stimulus to the user, the second output stimulus related to one or both of the ambient temperature of the user exceeding a first threshold and the level of noxious gas present within the helmet exceeding a second threshold.

18. The helmet according to claim 17, comprising a user interface configured to notify a centralised control facility, responsive to the second output stimulus indicating the ambient temperature of the user has exceeded the first threshold and/or the level of noxious gas present within the helmet has exceeded the second threshold.

19. An active welding helmet for a user welding components, the helmet comprising:
a welding helmet including a shading lens;
an indicator system for providing one or more of visual stimulus, audible stimulus, or tactile stimulus, to the user; and
a user interface configured to notify the user, via the indicator system, that a desired pre-weld temperature of the components to be welded has been reached.

20. The helmet according to claim 19, comprising;
a temperature sensor within the welding helmet and configured to detect an ambient temperature of the user; and
a noxious gas sensor within the welding helmet and configured to detect a level of noxious gas present within the helmet;
wherein the user interface is further configured to issue to the user, via the indicator system, a series of indications, with changes in urgency, related to increasing values of one or both of the ambient temperature of the user and the level of noxious gas present within the helmet.
